(19) United States
(12) Patent Application Publication
(10) Pub. No.: US 2014/0316467 A1
(43) Pub. Date: Oct. 23, 2014

(54) BONY STRUCTURE FIXATION CLAMP

(71) Applicant: NLT SPINE LTD., Kfar Saba (IL)

(72) Inventors: Tzony Siegal, Shoeva (IL); Oded Loebl, Tel Mond (IL); Didier Toubia, Raanana (IL)

(21) Appl. No.: 14/354,938

(22) PCT Filed: Oct. 31, 2012
(86) PCT No.: PCT/IB2012/056052

§ 371 Date: Apr. 29, 2014

Related U.S. Application Data
(60) Provisional application No. 61/553,284, filed on Oct. 31, 2011.
(60) Provisional application No. 61/620,499, filed on Apr. 5, 2012.
(60) Provisional application No. 61/652,346, filed on May 29, 2012.

(51) Int. Cl. A61B 17/70

(52) U.S. Cl. CPC A61B 17/7065
USPC 606/249

(57) ABSTRACT

A clamp comprises a first structure configured to exert force on a first bony structure at a first point of contact, a second structure configured to exert force on a second bony structure at a second point of contact, a spacer pivot positionable between the first and second bony structures and comprising a pivot point offset in an anterior (or posterior) direction relative to a line between the first and second points of contact when (i) the first structure is deployed to engage the first bony structure and (ii) the second structure is deployed to engage the second bony structure. The clamp may include a clamping structure, adjusting a distance between the first and second structures, and preferably also connecting the spacer pivot. The deployed spacer pivot is operatively connected to the first and second structures when the first and second structures are deployed.

### FIELD AND BACKGROUND OF THE INVENTION

There are various known implants and procedures for handling pain associated with degenerative spinal disk diseases. One procedure is interbody fusion, which is performed in the anterior portion of the body. Surgery on the anterior portion is generally more invasive than surgery on the posterior portion of the body, since the organs of the body are generally in the anterior portion. Surgical techniques performed in the posterior portion of the body utilize bone screws, such as pedicles or facets. However, there may be serious complications associated with pedicle screw fixation, including nerve root injuries caused by accidental screw insertion, spinal fluid leakage, nerve injury and infection. In general, this technique is invasive. Another disadvantage of this technique is radiation exposure of operators and patients, because X-ray images are taken during the surgery. On the other hand, bone screws, such as pedicle screws, have significant biomechanical strength. Spinous process fixation devices are implemented in the posterior of the spine—they have the advantage of minimizing tissue damage. However, such spinous process fixation devices used as anchors provide less biomechanical strength than pedicle screw fixation, although they reduce or eliminate complications such as spinal fluid leakage or nerve injury, and requires no excessive excision of the paraspinal muscles. In addition, the relatively low invasiveness of spinal instrumentation using the bony structures is an important advantage, because such method is associated with shortened operation time, reduced bleeding, and reduced length of subsequent hospital stay.

There is a compelling need for implants and procedures which are minimally invasive yet are stable and effective.

### SUMMARY OF THE PRESENT INVENTION

One aspect of the present invention is a clamp comprising a first structure configured to exert force on a first bony structure at a first point of contact; a second structure configured to exert force on a second bony structure at a second point of contact; and a spacer pivot configured to be positionable between the first and second bony structures and comprising a pivot point offset in one of an anterior and posterior direction relative to a line between the first and second points of contact when (i) the first structure is deployed to engage the first bony structure and (ii) the second structure is deployed to engage the second bony structure, the first and second structures connected one another directly or through an intermediate structure, the spacer pivot operatively connected to the first and second structures when the first and second structures are deployed at the respective first and second points of contact and the spacer pivot is positioned between the first and second bony structures.

A further aspect of the present invention is a method of clamping bony structures, comprising positioning a first structure so as to have a first point of contact on a first bony structure and positioning a second structure so as to have a second point of contact on a second bony structure; providing a connection between the first and second structures; placing a body spacer between a first bony structure and a second bony structure such that the body spacer comprises a pivot point and such that the pivot point is offset in one of an anterior and posterior direction relative to a line between the first and second points of contact when (i) the first structure is positioned on the first bony structure and (ii) the second structure is positioned on the second bony structure; and adjusting the first structure and/or the second structure so that the first structure forcibly hugs the first bony structure and so that the second structure forcibly hugs the second bony structure.

A still further aspect of the present invention is a clamp comprising a first structure configured to exert force on a first bony structure at a first point of contact; a second structure configured to exert force on a second bony structure at a second point of contact; and a spacer pivot configured to be positionable between the first and second bony structures and comprising a pivot point, the pivot point offset in one of an anterior and posterior direction relative to a line of force exerted by the first structure and relative to a line of force exerted by the second structure, when (i) the first structure is deployed to engage an upper part of the first bony structure and (ii) the second structure is deployed to engage a lower part of the second bony structure, the first and second structures connected to one another directly or through an intermediate structure, the spacer pivot operatively connected to the first and second structures when the first and second structures are deployed at the respective first and second points of contact and the spacer pivot is positioned between the first and second bony structures.

A yet still further aspect of the present invention is a multiple level clamp comprising a first structure configured to exert force on a first bony structure at a first point of contact; a second structure configured to exert force on a second bony structure at a second point of contact; a first spacer pivot configured to be positionable between the first bony structure and a third bony structure and comprising a first pivot point; a second spacer pivot configured to be positionable between the second bony structure and the third bony structure and comprising a second pivot point; a line between the first and second pivot points offset in one of an anterior and posterior direction relative to a line between the first and second points of contact when (i) the first structure is deployed to engage the first bony structure and (ii) the second structure is deployed to engage the second bony structure; and the first and second structures connected to one another directly or through an intermediate structure, the first and second spacer pivots operatively connected to the first and second structures when (i) the first and second structures are deployed at the respective first and second points of contact and (ii) the first and second spacer pivots are positioned respectively between the first and third bony structures and between the second and third bony structures.

These and other features, aspects and advantages of the present invention will become better understood with reference to the following drawings, descriptions and claims.

### BRIEF DESCRIPTION OF THE DRAWINGS

Various embodiments are herein described, by way of example only, with reference to the accompanying drawings, wherein:

FIG. 1 is an isometric view of a clamp, in accordance with one embodiment of the present invention;

FIG. 1A is an isometric view similar to FIG. 1 and showing a line between first and second points of contact, in accordance with one embodiment of the present invention;

FIG. 2 is a side view of the implant of FIG. 1 deployed clamping bony structures and showing planes of contact of the first and second hook and of the spacer pivot, in accordance with one embodiment of the present invention;

FIG. 3 is an end view of a deployed implant of FIG. 1 showing an end view of the spacer pivot, showing forces exerted on the first and second hooks and showing a pivot point of the spacer pivot, in accordance with one embodiment of the present invention;

FIG. 4 is an isometric view of a clamp whose first and second structures have heads comprising concavities for the holding rod, in accordance with one embodiment of the present invention;

FIG. 4A is an isometric view of a clamp wherein the head of one of the first and second structures has a concavity, in accordance with one embodiment of the present invention;

FIG. 5 is an isometric view of a clamp similar to FIG. 4 except with vertically oriented heads with concavities, in accordance with one embodiment of the present invention;

FIG. 6 and FIG. 7 are isometric views of a clamp utilizing a rod with a slit and utilizing movable hooks, in accordance with one embodiment of the present invention;

FIG. 8 is an isometric (partially schematic) view of a clamp with multiple spacer bodies, in accordance with one embodiment of the present invention;

FIGS. 9-12 are isometric views of a clamp utilizing a ratchet and tooth mechanism, in accordance with one embodiment of the present invention;

FIG. 13 is an isometric view of a clamp whose first and second hooks comprise a plurality of hingedly interconnected segments in a straightened configuration, in accordance with one embodiment of the present invention;

FIG. 14 is an isometric view of the clamp of FIG. 13 wherein the first and second hooks are in a curved deployed state, in accordance with one embodiment of the present invention;

FIG. 15 is a flow chart of a method, in accordance with one preferred embodiment of the present invention;

FIG. 16 is an isometric view of a structure of a published coassigned application showing a plurality of segments utilizable in first and second hooks of a clamp, in accordance with one embodiment of the present invention;

FIG. 17 is a partial isometric view of a tightening element used in conjunction with the plurality of segments shown in FIG. 16;

FIG. 18 is an isometric view of a deployed clamp clamping adjacent spinous processes, in accordance with one embodiment of the present invention;

FIG. 19 is an isometric view of a deployed clamp clamping adjacent transverse processes, in accordance with one embodiment of the present invention; and

FIG. 20 is an isometric view from the side of the clamp of FIG. 19.

### DETAILED DESCRIPTION OF THE INVENTION

The following detailed description is of the best currently contemplated modes of carrying out the invention. The description is not to be taken in a limiting sense, but is made merely for the purpose of illustrating the general principles of the invention, since the scope of the invention is best defined by the appended claims.

The present invention generally provides a method and apparatus for a bony structure fixation clamp. Typically, such a clamp may be applied to the lumbar (or cervical) portion of the spine, which has an inward curvature, although this is not a limitation. First and second hooks of the clamp may be configured to exert force on first and second bony structures, typically but not necessarily spinous processes, which may be adjacent spinous processes, at first and second points of contact. “First and second bony structures” are specifically defined herein such that one bony structure, for example the first bony structure, is anatomically superior (upward, toward the head) and the other bony structure, such as the second bony structure, is inferior, using anatomical orientation. This is the orientation, for example, of adjacent spinous processes, a type of bony structure. A spacer pivot may be configured to be positionable between the first and second bony structures. The spacer pivot may comprise a pivot point that is offset relative to a line between the first and second points of contact when (i) the first structure (i.e. first hook) is deployed to engage the first bony structure and (ii) the second structure (i.e. second hook) is deployed to engage the second bony structure. The spacer pivot's pivot point may be offset in either the anterior direction (or in other preferred embodiments in the posterior direction) relative to a line connecting between the first and second points of contact (or relative to lines of force exerted by the first and second structures (i.e. first and second hooks) against the bony structures). The first and second structures may be connected directly, such as having by a rod projecting from one hook fit into a concavity in the head of a second hook, or through an intermediary structure, such as a rod. A clamping structure may connect the first and second hooks and may adjust a distance between the first and second hooks. In preferred embodiments the clamping structure may also connect the spacer pivot. When the spacer pivot is offset toward the anterior of the body, tightening the clamp and reducing the distance between the hooks may put adjacent vertebrae at a lordotic angle. The hooks may be connected to the clamping structure by virtue of a rod and concavities in heads of the spacer body and in the head of at least one of the hooks. In another version, the hooks may have a distance between them in at least one dimension adjusted by a ratchet and tooth mechanism. In one preferred embodiment, each of the first and second structures is segmented and comprises non-linear technology such as described in U.S. Patent Pub. No. 2010/0198263.

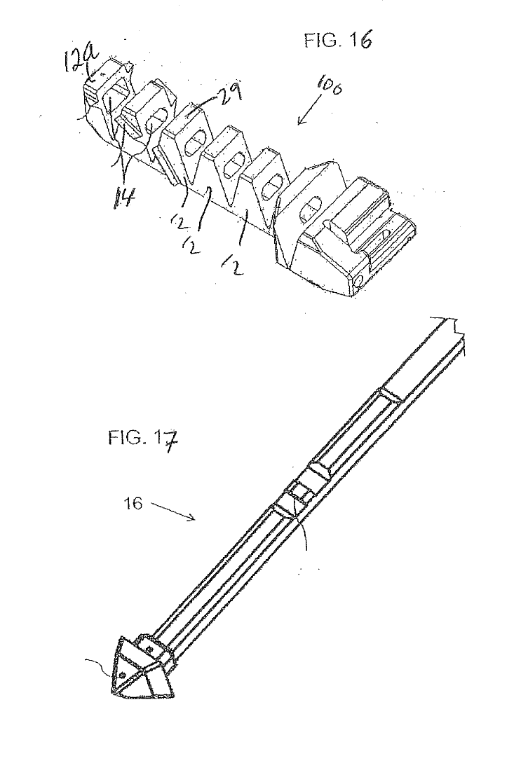

In contrast to prior art implants that use bone-screw based fixation devices, the present invention may use an implant functioning as a bony structure fixation clamp. In contrast to prior art procedures for handling pain associated with degenerative spinal disk diseases, in which two or more adjacent vertebral bodies are fused together, the present invention may fixate and/or fuse together adjacent bony structures such as spinous processes. This may eliminate the need for access to the anterior portion of the spine and may eliminate the need for a medical device (such as an intravertebral body device) to be used as an adjunct for fixation and/or fusion of adjacent vertebral bodies. In contrast to the prior art devices and procedures for handling pain associated with degenerative spinal disk diseases, the present invention may utilize a surgical procedure that accesses the body of the subject on only one side, for example using a Wiltse approach. By having the whole surgical procedure on only one side of the body, as opposed to on two sides, tissue trauma and destruction of subperiosteal dissection may be significantly reduced (including from any bone grafting). In contrast to conventional prior art bone-screw-based fixation techniques, the clamp of the present invention may not violate the precious cortex of any posterior element. In further contrast to prior art spinous process fixation clamps, in which the body spacer is in the same vertical plane as the hooks, the present invention may utilize a body spacer that is in a different vertical plane than the vertical plane that the hooks are in. This may allow the clamping force applied by the hooks to pivot on the body spacer. As a result, a lordotic angle may be achieved between adjacent vertebrae, which is consistent with the inward curvature of the healthy spine. By employing polyaxial heads (i.e. “tulip” shaped heads), the components may be free to move until being locked by a locking screw. This reduces the stresses on the device and therefore on the bone itself. In further contrast to the prior art bony structure fixation clamps such as spinous process fixation clamps, in which the body spacer is in the same vertical plane as the hooks, the implant and method of the present invention may create far less internal stress on the bony structure fixation clamp itself. This is because as a result of the ability of the body spacer to pivot, the rod connecting the hooks can be conveniently slipped into a “tulip” type head for holding the rod, in certain preferred embodiments. In further contrast to the prior art implants and methods for handling pain associated with degenerative spinal disk diseases, which are either too invasive or which lack biomechanical strength, the implant and method of the present invention may be minimally invasive as a result of utilizing a spinal process fixation clamp, yet may be of sufficient biomechanical strength since the clamp may embrace the bony structure at the far ends against the strongest bone resistance, namely the cortical bone of the spinal processes. An added advantage of this may be that the bony structures are not weakened by drilling into and/or through them. In further contrast to the prior art, which discloses a segmented implant body that by use of a tensioning element can be converted from a straightened state to a curved deployed state, but where the entire plurality of segments is deployed in the curved state as an implant, not as a clamp, the present invention, in one preferred embodiment, discloses a clamp comprising a first hook and/or a second hook that may be made of a plurality of segments convertible to a curved state from a straightened state. In particular, in contrast to the prior art segmented implant body, in which the tops of the segments are not specifically designed to clamp or to support a relatively large load, but rather the substantially flat side surfaces of the segments (i.e. the surfaces that engage the spinous processes) sustain the load, in the present invention, the “tops” (see 29 in FIG. 16 corresponding to 29 in FIG. 14 but in FIG. 14 the orientation is such that the “tops” appear on the side) of the segments in the plurality of segments that may form the structures (i.e. hooks) may be used to exert clamping forces on each of adjacent bony structures, which may require supporting a relatively large load.

The principles and operation of an apparatus and method utilizing bony structure fixation clamps according to the present invention may be better understood with reference to the drawings and the accompanying description.

The drawings show various views of an implant functioning as a bony structure fixation clamp, as an alternative to bone-screw based fixation techniques.

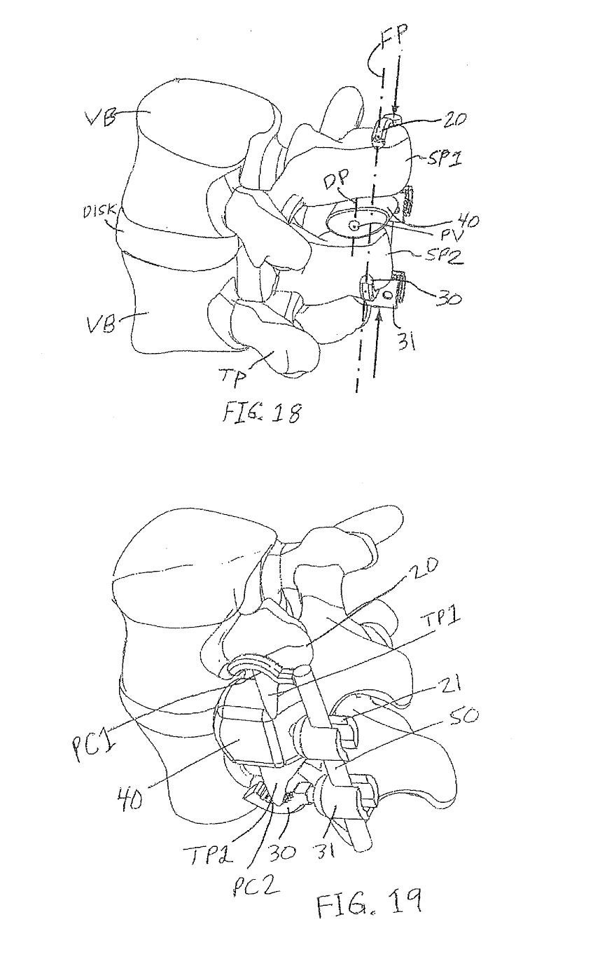

FIG. 1 shows a clamp 10 comprising a first structure 20, which may be a first hook 20 (in any preferred embodiment of this patent application), configured to exert force on a first bony structure, which may be the upper part of a first spinous process, SP1, at a first point of contact PC1. Also shown is a second structure 30, which may be a second hook 30 (in any preferred embodiment of this patent application), configured to exert force on a second bony structure, which may be the lower part of a second, and preferably an adjacent, spinous process SP2, at a second point of contact PC2. Generally, as shown in FIG. 18, the first structure 20 may be deployed to engage the first (or upper) bony structure SP1 (for example at an upper part of the first bony structure and second structure 30 may be deployed to engage the second (or lower) bony structure SP2 (for example at a lower part of the second bony structure).

Clamp 10 may also have a body spacer, also called a “spacer body” or a “spacer pivot” 40 configured to be positionable between the first and second bony structures SP1, SP2. Spacer pivot 40 may include a pivot point PV (FIG. 3) with respect to the opposing forces exerted on the respective first and second points of contact on the respective first and second structures 20, 30. In other words, body spacer 40 is configured to be positionable such that when (1) the first structure 20 is deployed to engage the first bony structure and (ii) the second structure 30 is deployed to engage the second bony structure, pivot point PV may be offset in an anterior direction (or in certain other preferred embodiments in a posterior direction) relative to a line L between the first and second points of contact. The forces (shown by the straight arrows F1, F2 in FIG. 3) exerted by the first and second structures 20, 30 are in the vicinity of the line L between the first and second points of contact. A torque T (FIG. 3) is created on the pivot point PV, as shown by the curved arrow in FIG. 3, when the forces are exerted on the respective first and second structures 20, 30.

FIG. 18 shows a clamp 10 of the present invention deployed so as to clamp adjacent spinous processes SP1, SP2. Adjacent vertebral bodies (VB) and the intermediary disk (DISK) between the vertebral bodies are also shown. As shown in FIG. 18, first and second structures 20, 30 (which in this embodiment are hooks 20, 30) may lie on a first plane FP (which is a “first vertical plane” when the clamp is deployed) and the pivot point PV of body spacer 40 may lie on a more distal plane DP in relation to a user standing behind the posterior of the patient/subject. This more distal plane may be offset in relation to the first plane FP that first and second structures 20, 30 may lie on. In this embodiment, the offset is in the anterior direction anatomically. First structure 20 (which may be a first hook 20) may be configured to and may exert force against the first bony structure SP1 at a first point of contact (called PC1—see FIG. 1) and likewise second structure 30 (which may be a hook 30) may be configured to and may exert force against a second bony structure SP2 at a second point of contact (called PC2—see FIG. 1). FIG. 18 also depicts pivot point PV as offset in relation to a line (lying on first plane FP) between first and second structures 20, 30 and in particular in relation to a line between inside surfaces of first and second structures 20, 30 at first and second points of contacts of first and second structures 20, 30 (the first and second points of contact PC1, PC2 are not visible in FIG. 18).

In any preferred embodiment, body spacer 40 may be configured to be positionable such that pivot point PV is offset (or also offset) in an anterior direction (or for the above-mentioned certain other preferred embodiments in a posterior direction) relative to lines of force exerted by first structure 20 (exerted downwardly for example against first bony structure SP1 as shown in FIG. 18) and relative to lines of force exerted by second structure 30 (exerted upwardly for example against second bony structure SP2 as shown in FIG. 18), when (i) the first structure 20 is deployed to engage the first bony structure and (ii) the second structure 30 is deployed to engage the second bony structure. The lines of force exerted by each of the first and second structures 20, 30 are shown by the two straight arrows F1, F2 in FIG. 3 pointing in opposite directions.

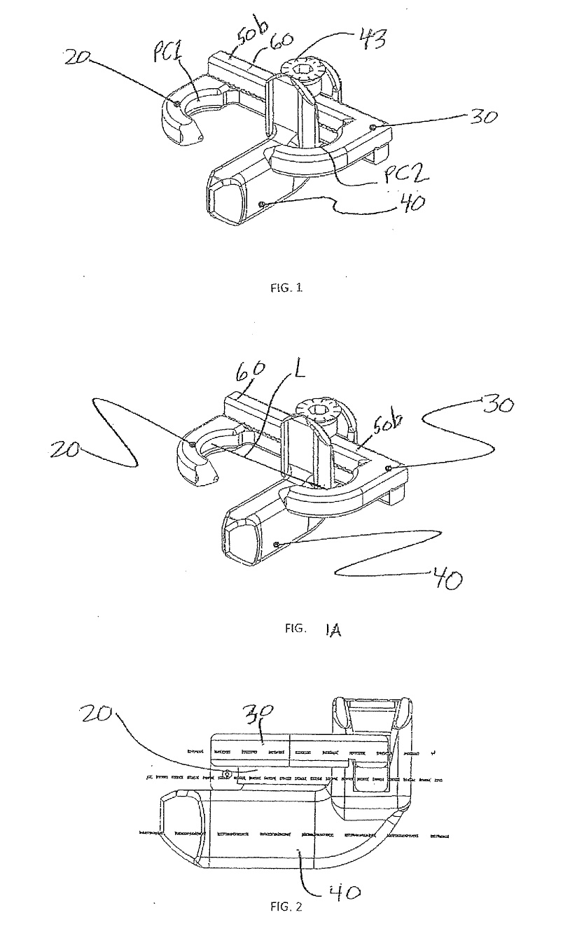

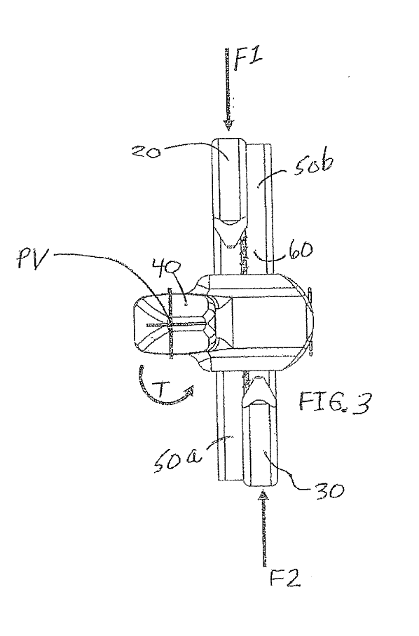

It is noted that FIGS. 1-3 depict a preferred embodiment of a clamp 10 whose first structure (i.e. hook) 20 is slightly offset from the second structure (i.e. hook) 30 in the direction of the spacer pivot 40. For example, a clamp 10 as shown in FIGS. 1-3 is deployed to clamp spinous processes or other bony structures in the positions shown in FIGS. 2-3, the first structure 20 may lie on a first vertical plane that is slightly more anterior, i.e. distal in relation to a user standing behind the posterior of the subject's body, than the second vertical plane that second structure 30 lies on. In such a case (where first and second structures 20, 30 do not lie on the same vertical plane) the spacer body 40 may be said to lie on a third vertical plane offset from both the first and second vertical planes in an anterior direction. In other preferred embodiments, however, for example in the embodiment shown in FIG. 18 (and the embodiment shown in FIG. 20), the first and second structures 20, 30 may lie on the same vertical plane. In this context, and in general in this patent application, the terms “vertical” and “vertical plane” as used herein refer to the orientation in either a sagittal plane, a plane parallel to the sagittal plane, a frontal/coronal plane or a plane parallel to the frontal/coronal plane. A distal vertical plane is defined to be a vertical plane that is more anterior than another vertical plane (the other vertical plane thereby being more proximal). A vertical plane “displaced” from another vertical plane means displaced as measured along an imaginary line running between the two vertical planes, the line being transverse to the two planes.

In any of the preferred embodiments of the present invention, the first and second points of contact PC1, PC2 between a first or second structure 20, 30 (which may be a first or second hook) and a bony structure are defined to include a case where an intermediary material interposes between one or more of the first or second structures 20, 30 and the first and second bony structure and there is no absolute direct physical contact between one or more of the first and second structures and the first and second bony structure. The intermediary material may be part of a device or may be human tissue that is not technically part of the bony structure.

In addition, in any of the preferred embodiments of the present invention, the forces exerted by first and second structures 20, 30 need not necessarily be applied at a single point on each bony structure. Rather, there may be multiple points of contact in a region of contact. Accordingly, the first structure 20 or second structure 30 may be configured to exert force on a first or second bony structure at a point of contact or at many points of contact in the region of contact. Where contact occurs at multiple points in a region of contact, the phrase “point of contact” is used herein in the description and claims to refer to the geometrical center (centroid) of the region of contact. Furthermore, if there are multiple regions of contact, the “point of contact” that should be used for the purpose of all geometrical definitions is the centroid of the contact region at the superior edge of the upper spinous process (for the first point of contact PC1) and the centroid of the contact region at the inferior edge of the lower spinous process (for the second point of contact PC2). If the bony structure is not a spinous process, then an analogous centroid is used for the geometrical definitions so as to refer to the part of the structure 20, 30 best positioned to apply the clamping force. The region of contact may typically be on the underside or inward-facing surface of first and second structures 20, 30, which in some embodiments is curved inwardly, as shown for example in FIG. 1. When the clamp 10 is deployed to clamp bony structures, the “underside” of the second structure (which may be a lower hook) 30 may actually be located above (superior to) the outside surface of the second structure 30.

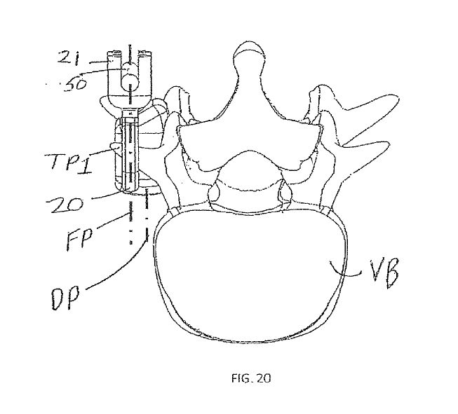

Typically, the pivot point of spacer pivot 40 is offset in an anterior direction (or in a posterior direction in certain other preferred embodiments) relative to any point along line L. This is because typically, the pivot point is offset in the anterior direction (or in the posterior direction in certain other preferred embodiments) relative to both the first and second points of contact and relative to whichever point of contact among the first and second points of contact is the more anterior. In a typical application where the clamp 10 is deployed so as to engage two or more spinous processes, SP1, SP2, as shown in FIG. 18, (or two or more transverse processes TP1, TP2 as shown in FIGS. 19-20) the offset may be in the anterior direction.

As far as the quantitative amount of the offset is concerned relative to the line L connecting the points of contact (or in other preferred embodiments relative to all points on such a line L) or in other preferred embodiments relative to the lines of force exerted by the first and second structures 20, 30, the pivot point PV of spacer pivot 40 may be offset relative to the line (or all points on the line) or line of force by at least a half a centimeter, or in other preferred embodiments, by between half a centimeter and one centimeter, or in other preferred embodiments by between half a centimeter and two centimeters, or in still other preferred embodiments, by between half a centimeter and one and a half centimeters. In cases, where the bony structures are not spinous processes, for example transverse processes or pedicles, the offset may be of similar magnitude. These magnitudes and ranges of magnitudes are not limited to one particular embodiment only and may be applied to any preferred embodiment.

As a result of the fact that the clamp 10 may utilize a spacer body or spacer pivot 40 that is offset from the line between first and second points of contact (or and/or offset from lines of force F1, F2 exerted by first and second structures 20, 30) when first and second structures 20, 30 are deployed to engage, i.e. clamp the respective upper and lower bony structures, clamp 10 may be adapted to clamp adjacent vertebrae at a lordotic angle. This is consistent with the inward curvature of the lumbar and cervical portions of the healthy spine.

In other preferred embodiments, any of the embodiments of the clamp of the present invention may be used to clamp bony structures at a kyphotic angle. That is, the clamp and method herein described may be used for parts of the spine having a kyphotic angle rather than parts of the spine having a lordotic angle (or for other bony structures as defined herein).

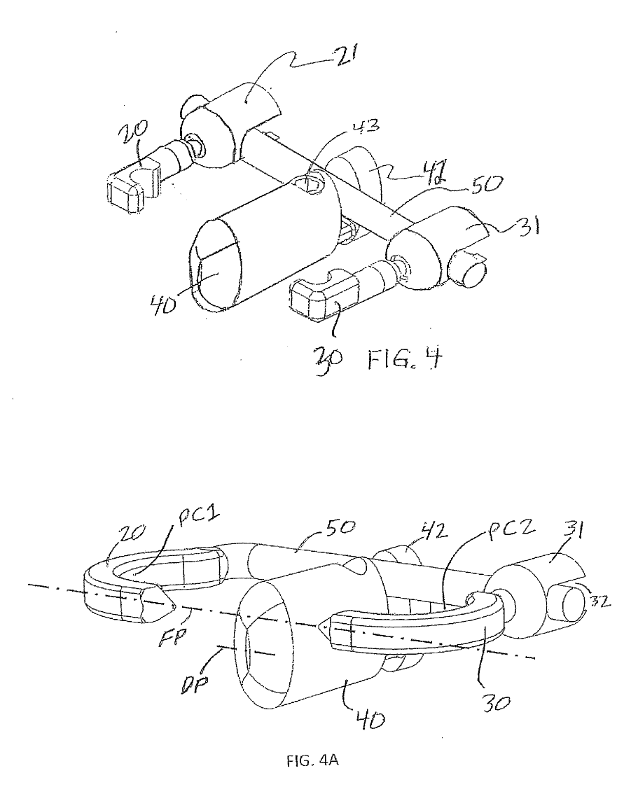

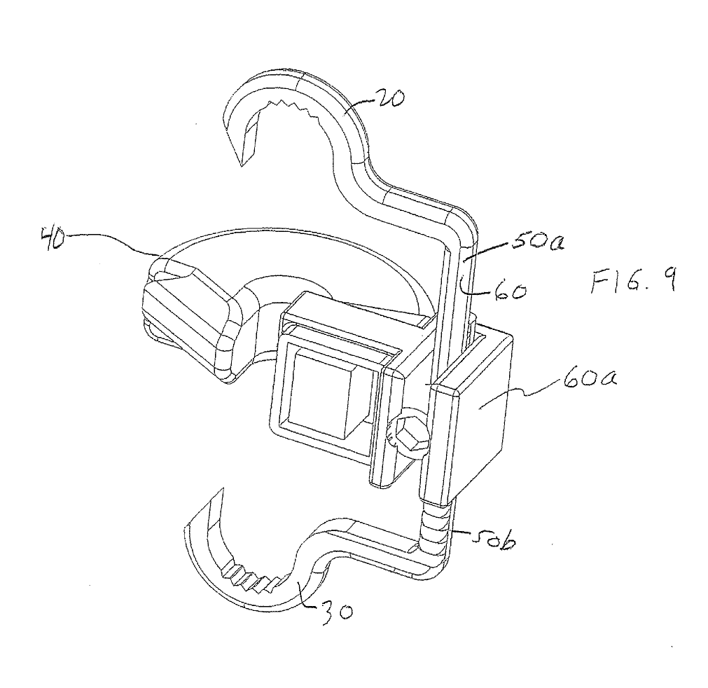

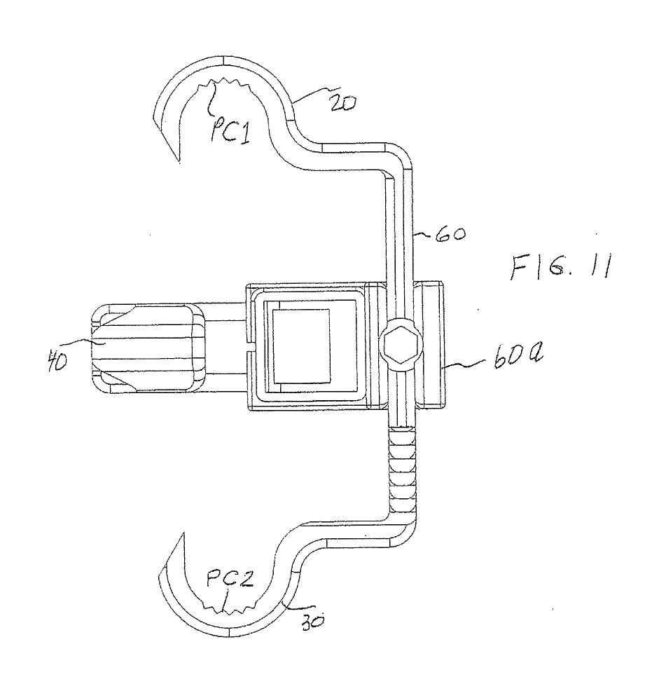

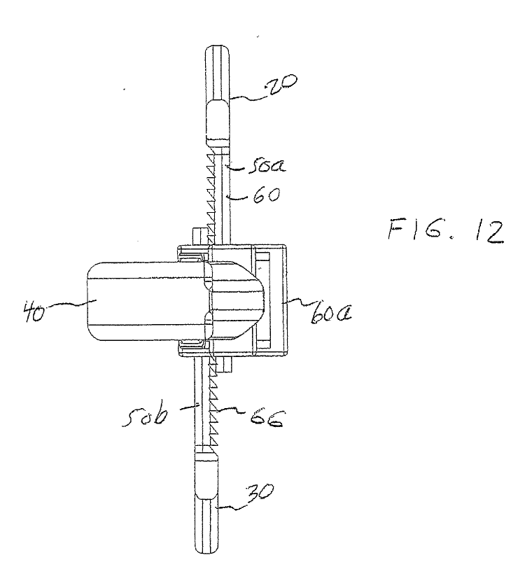

The first and second structures 20, 30 may be connected to one another directly or through an intermediate structure. For example, as shown in FIGS. 4, 5, 13, 14, a rod 50 may connect first and second structures 20, 30. In other preferred embodiments, for example FIGS. 1, 2, 3, 4a, 9-12, 19, a rod 50 may be integrally formed with and may project from one or both of the first and second structures 20, 30 and this rod 50 may connect directly to another of the first and second structures. In the case of FIGS. 9-12, each of the first and second structures 20, 30 may have a rod 50a, 50b projecting therefrom, and these rods 50a, 50b may interact by having mating teeth.

In the case where first and second structures 20, 30 are not directly connected but are connected through an intermediary structure, the intermediate structure may for example be a rod 50 between first and second structures 20, 30, as shown in FIGS. 4, 5, 13-14. The intermediary structure may also be a clamping structure 60 that may connect first structure 20 to second structure 30. Clamping structure 60 may include a tightening mechanism 60a (see FIG. 9) to adjust a distance between the first and second hooks 20, 30 in at least one dimension. Clamping structure 60 may also include a compression instrument (not shown) to compress/adjust a distance between first and second structures 20, 30 that are connected by a rod 50, for example in the embodiments shown in FIGS. 4, 5, 13, 14.

In preferred embodiments, the spacer pivot 40 may also be connected to the intermediate structure (such as a rod 50 or a clamping structure 60) between first and second structures 20, 30 or may also be connected to a rod 50 projecting from one of the first and second structures 20, 30. In certain other preferred embodiments, the spacer pivot 40 is not physically connected to the other parts of the clamp 10 (i.e. the first and second structures 20, or a rod 50 or clamping structure 60) but when the spacer pivot 40 is positioned between the bony structures SP1, SP2, and offset as described, the spacer pivot functions as part of the clamp 10. Accordingly, and as a result of the pivot point PV of spacer pivot 40 being offset as described herein, spacer pivot 40 may be operatively connected to first and second structures 20, 30 when first and second hooks 20, 30 are deployed at the respective first and second points of contact and spacer pivot 40 is positioned between the first and second bony structures (such as first and second spinous processes). The operative connection and functional relationship may then be implemented when clamping forces are exerted by first and second structures 20, 30.

The preferred embodiment shown in FIGS. 1-3 for clamp 10 may also be implemented with connecting elements or clamping structures described in connection with the other preferred embodiments described herein. For example, although FIG. 1 happens to illustrate a clamp 10 with a ratchet and tooth mechanism, in fact the embodiment shown in FIGS. 1-3 may incorporate the connecting elements shown in FIG. 4 and FIG. 4a where one or more of first and second structures 20, 30 have polyaxial heads with concavities (tulip type heads), may be used with body spacer 40.

In general, any suitable structural features or steps shown in one of the preferred embodiments described herein may be incorporated into other preferred embodiments described in any method or clamp described in this patent application. This is also true regarding any of the means described herein for connecting the first and second structures 20, 30 and the means described herein for adjusting distances between such first and second structures 20, 30 and any clamping structures, rods or other connecting elements, whether integrally formed as part of the clamp or method or whether utilized as an external compression or other instrument.

As shown in FIGS. 4-12, there are various ways in which the first and structures 20, 30 and spacer 40 may be configured. For example, as shown in FIG. 4, body spacer 40 may have a locking plate 42 that may have a concavity for receipt of rod 50. Another type of locking component may be a polyaxial head having a concavity which is sometimes referred to as a tulip type head. One or both of the first and second structures (for example hooks) 20, 30 may have a head, for example head 21 in the case of the head of the first structure 20 and head 31 in the case of the second structure 30, comprising a concavity or may be a polyaxial head (tulip type head).

In an alternative version, shown in FIG. 4A, in which only one of the first and second structures 20, 30 comprises a polyaxial head, first structure 20 may have an integrally joined rod 50 projecting from first structure 20 and second structure 30 may have a polyaxial or tulip type head 31. In addition, body spacer 40 may have a locking plate 42 that may be adjacent a body spacer concavity. Accordingly, only one of the first and second structures, namely second structure 30, may have a polyaxial head 31 that comprises a concavity 32. The other structure (i.e. hook) of the first and second structures 20, 30, namely first hook 20 may have a rod 50 integrally connected thereto. The rod 50 may be configured to fit into the spacer concavity at the end of the spacer 40 such that rod 50 may simultaneously be slipped into the concavity in the head of second hook 30. This may be used to position the clamp 10 to be tightened and used. As shown in FIG. 4A, first structure 20 may be configured to exert force against the first bony structure SP1 at a first point of contact PC1 and likewise second structure 30 may be configured to exert force against a second bony structure SP2 at a second point of contact PC2. As seen from FIG. 4A, pivot point PV may be offset in relation to a line (on first plane FP) between structures 20, 30 and in particular between first and second points of contacts PC1, PC2.

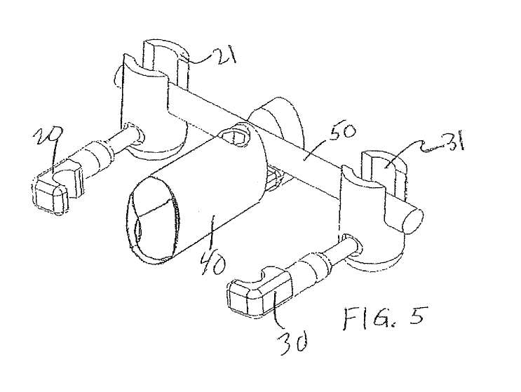

FIG. 5 shows an embodiment similar to FIG. 4, except where the polyaxial heads 21, 31 of the hooks 20, 30 are oriented perpendicularly to the length of the hooks, i.e. vertically as shown in the figure. It is noted that FIG. 4 and FIG. 5 illustrate ways in which the first and second structures may be connected and versions of a clamping structure; these figures are not intended to accurately illustrate the offset of pivot point PV (which is more accurately illustrated for example in FIG. 1-4A, 13-14, 18, 20).

Clamping structure 60 may be used for tightening and loosening (i.e. clamping) the first structure 20 to the first bony structure and for tightening and loosening the second structure 30 to the second bony structure by adjusting the distance between the first and second hooks 20, 30 in at least one dimension. In one preferred embodiment, for example in FIGS. 9-12, clamping structure 60 may include a ratchet and tooth mechanism. In other preferred embodiments in which one or more concave polyaxial heads (i.e. tulip type heads) and a rod are used, the clamping structure 60 may include a separate external instrument outside the body of the patient (not shown) that is used to adjust the distance between the first and second structures 20, 30. For example, if both of the first and second structures 20, 30 have a concave or polyaxial head, then a compression instrument outside the body may be used to tighten or loosen, and thereby adjust the distance between, the first and second structures 20, 30. Alternatively, if only one of the hooks has a concave or tulip type head and the other hook is connected to an integrally attached rod, a rod holder outside the body may be used to adjust the distance between the hooks.

In one preferred embodiment, for example as shown in FIGS. 1, 9-12, the spacer pivot 40 may be mounted on the clamping structure 60. In certain other preferred embodiments, the spacer pivot 40 is not physically connected to the hooks or clamping structure but while positioned between the first and second bony structures functions as part of clamp 10. In still other preferred embodiments, for example those shown in FIGS. 1, 4A, 19, the body spacer 40 may be connected to at least one of the first and second structures 20, 30 (for example where one of the first and second structures has a projecting rod 50). In FIG. 1, the body spacer is connected to the rod 50 and secured by means of a locking screw 43. In the embodiment shown in FIG. 4A, the body spacer may be connected to the rod 50 by virtue of body spacer 40 itself having a locking plate 42 that is adjacent a concavity. Body spacer 40 may be connected in such a manner that the distance between the spacer body 40 and the first or second structure 20, 30 to which body spacer 40 is connected can be adjusted.

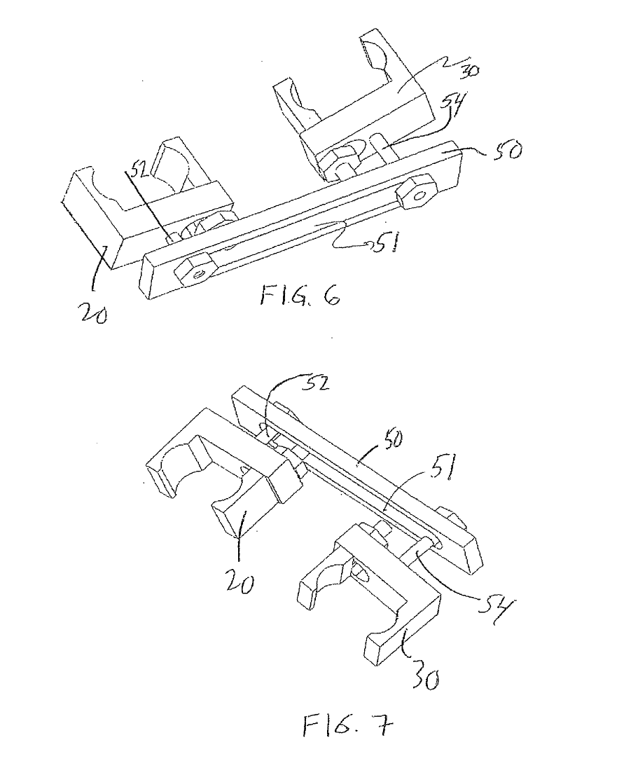

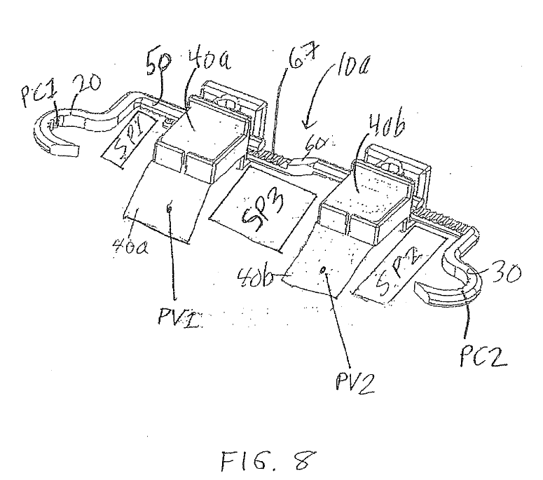

In accordance with the embodiment shown in FIGS. 6-7, instead of hooks the device shows first hook pair 20a and second hook pair 30a. Clamping structure 60 may include a rod 50 connecting the first and second hook pairs 20a, 30a. Each of the first and second hooks 20, 30 within each pair may be movable and lockable in place after positioning. In addition, the hook pairs themselves may be movable one with respect to the other. For example, first hook pair 20a may be connected to rod 50 by a first axis 52 and the first hook pair 20a may be rotatable around the first axis 52. Likewise, second hook pair 30a may be connected to rod 50 by a second axis 54 and the second hook pair 30a may be rotatable around the second axis 54. As seen from FIGS. 6-7, rod 50 may have a slot 51 to which the first axis 52 and second axis 54 are connected, allowing adjustment of the distance between the first and second hook pairs 20a, 30a. Accordingly, the first and second axes 52, 54 may be movable relative to one another within slot 51 to position the hooks at the optimal distance. Instead of a first hook clamping an upper part of a first bony structure and a second hook clamping a lower part of a second bony structure, in this embodiment, the upper and lower parts of the bony structure may be clamped by the first pair of hooks 20a and the upper and lower parts of the second bony structure may be clamped by the second pair of hooks 30a. Accordingly, although this embodiment does not include a spacer pivot, the bony structures may be pivoted by clamping both of them individually.

Furthermore, the preferred embodiments of FIGS. 1-5, 8-19 may be modified to incorporate the concept of FIGS. 6-7. Accordingly, any of these embodiments may have the rod 50 connecting the first and second structures 20, 30 comprise a slot 51 into which the first and second structures 20, 30 are fitted. This way, the distance between the first and second structures 20, 30 may be adjusted by moving or sliding the first and/or second structures 20, 30 through slit 51. The first and second structures 20, 30 may fit into slit 51 directly or by means of respective first and second axis 52, 54.

Instead of clamping two adjacent bony structures, such as adjacent spinous processes, it may be useful to clamp three adjacent bony structures. FIG. 8 shows a multiple level application of the clamp 10 of the present invention in which two body spacers 40a, 40b are used with first and second structures 20, 30 to clamp three (rather than two) bony structures together. As shown in FIG. 8, two spacer bodies 40a, 40b shown partially schematically may be used between three bony structures SP1, SP2, SP3 (the bony structures shown in boxes).

Accordingly, one preferred embodiment of the present invention is a multiple level clamp comprising a first structure 20 configured to exert force on a first bony structure SP1 at a first point of contact, a second structure 30 configured to exert force on a second bony structure SP2 at a second point of contact. The multiple level clamp 10a may also include a first spacer pivot 40a configured to be positionable between the first bony structure and a third bony structure SP3, the first spacer pivot comprising a first pivot point PV1. Note that in this “multi-level” embodiment, unlike all other embodiments herein, the second bony structure SP2 refers to the bony structure at the end of the group of three bony structures whereas the third bony structure SP3 is the bony structure in the middle of the three body structures. This labeling allows the “second” structure 30 of clamp 10a to be the one that exerts force on the “second” bony structure SP2). The multiple level clamp 10a may also include a second spacer pivot 40b configured to be positionable between the second bony structure SP2 and the third bony structure SP3. Second spacer pivot 40a may comprise a second pivot point PV2. Bony structures SP1, SP2, SP3 are depicted schematically in FIG. 8. In addition, spacer pivot and second spacer pivot 40a, 40b are depicted schematically since the extension of spacer pivot 40a and of second spacer pivot 40b are represented by a box in which the pivot point (PV1, PV2 respectively) is located.

It can readily be seen that a line between the first and second pivot points PV1, PV2 may be offset in an anterior direction (or in certain other embodiments in a posterior direction) relative to a line between the first and second points of contact PC1, PC2 when (i) the first structure is deployed to engage the first bony structure SP1 and (ii) the second structure is deployed to engage the second bony structure SP2. Clamp 10a is shown to include a clamping structure 60 that includes a rod 50 connecting the first and second structures 20, 30 and a ratchet and tooth mechanism 67. However, any other embodiment described herein for connecting first and second structures 20, 30 may be used. For example, instead of the ratchet and tooth mechanism shown in FIG. 8, one or more of first and seconds structures may have a polyaxial head and a rod 50 may connect them, in accordance with any of the embodiments described herein (for example, a rod projecting from one of first and second structures 20, 30 may connect to a polyaxial head of the other one of first and second structures 20, 30 or else a rod 50 may connect first and second structures 20, 30 each of which have polyaxial heads) As with other preferred embodiments described herein first and second spacer pivots 40a 40b may be connected to a rod 50 or clamping structure 60. The first and second spacer pivots PV1, PV2 may be operatively connected to the first and second structures 20, 30 when (i) the first and second structures 20, 30 are deployed at the respective first and second points of contact and (ii) the first and second spacer pivots 40a, 40b are positioned respectively between the first and third bony structures SP1, SP3 and between the second and third bony structures SP2, SP3.

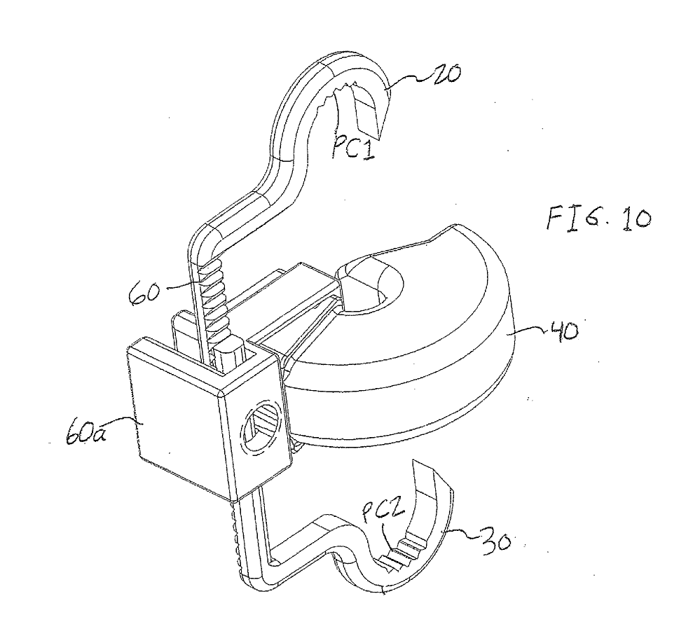

In another embodiment of the structure of clamping structure 60 and first and second structures 20, 30, shown in FIGS. 9-12, a clamping structure 60 may be defined to include the rods 50a, 50b that project from each of first and second structure 20, 30. Clamping structure 60 may also a ratchet and tooth mechanism (see FIGS. 10-12) including teeth 66 on rods 50a, 50b enabling the first and second structures 20, 30 to move relative to one another and to lock in place with the help of tightening mechanism 60a. The ratchet of the ratchet and tooth mechanism is not shown specifically but is well known in the art. Accordingly, in FIG. 1 and FIG. 1A and FIG. 3 the rod projecting from first structure 20 has been labeled “50a”, the rod projecting from second structure 30 has been labeled “50b” and the clamping structure 60 has been labeled “60”.

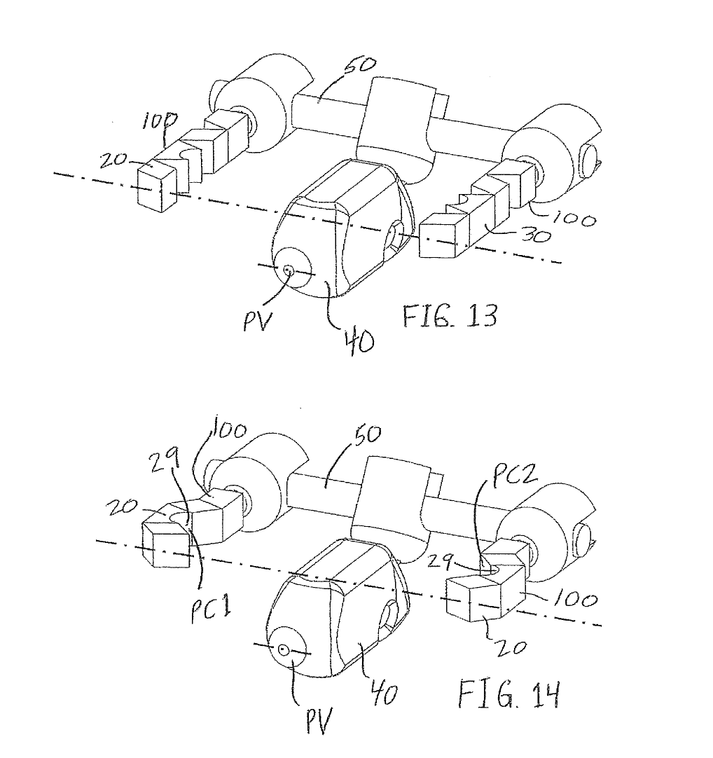

As seen in FIGS. 13-14, each of first and second hooks 20, 30 may comprise a plurality of segments 100 (the plurality taken as a whole is labeled “100”) which may be bingedly interconnected so as to assume a straightened state for insertion and a curved deployed state for use in the clamp 10. A channel 14 may pass from a distal one of said segments 12 along a majority of a length of the plurality of segments 100. Each of the first and second structures (which may be hooks) 20, 30 may also comprise an elongated tightening element 16 that may be anchored at the distal segment 12a of the plurality 100 and may pass along the channel 14 such that tension applied by tightening element 16 tends to bias the plurality of segments from the straightened state to the curved deployed state. It should be understood that in certain preferred embodiments only one of the first and second structures 20, 30 is comprised of the above-described plurality of segments hingedly interconnected and the other is a curved material not made of segments.

In the curved deployed state, the surfaces of the first and second structures 20, 30 that may have the first and second point of contact PC1, PC2 and that may exert clamping forces on the first and second bony structures SP1, SP2 (and in most preferred embodiments, make contact with the first and second bony structures), may be the sides 29 of the segments 12 of the plurality of segments 100.

As shown in FIGS. 16-17 of the prior art, the plurality of segments (taken as a whole 100) and the tightening element 16 may be configured to provide a locking arrangement such that, when the tightening element 16 is deflected to reach the curved deployed state, the locking arrangement is effective to lock the tightening element relative to said implant body, thereby retaining said implant in said curved deployed state. This is described in further detail in US Patent Application Publication No. 2010/0198263 having a publication date of Aug. 5, 2010. This patent publication is incorporated herein by reference, and includes details of the structure of an implant body which may be used to comprise one or more of the first and second hooks of the clamp, as described herein.

In the preferred embodiment shown in FIGS. 13-14, as well as the other preferred embodiments described in this patent application, the pivot spacer 40 is most preferably implemented as a structure according to US Patent Application Publication No. 2010/0198263 having a publication date of Aug. 5, 2010. This patent publication provides further details of the structure of the spacer 40. For example, pivot spacer 40 may be comprised of a plurality of segments (similar to the structure shown in FIG. 16) which may be hingedly interconnected so as to assume a straightened state for insertion and a curved deployed state for use in the clamp 10. A channel may pass from a distal one of said segments of spacer 40 along a majority of a length of the plurality of segments. An elongated tightening element may be anchored at the distal segment and may pass along the channel such that tension applied by a tightening element tends to bias the plurality of segments from the straightened state to the curved deployed state.

In one preferred embodiment, after insertion of the pivot spacer 40 between two adjacent bony structures, the two clamp arms are inserted above the superior process and below the inferior process, in a manner similar to conventional hook-tipped needles. The clamps are then manipulated for insertion into the tightening and locking mechanism 60a (see FIGS. 9-12) where they are closed around the spinous processes to hold them against the spacer 40. A tightening mechanism may be actuated by turning a suitable key or actuator, as is known in the art, or tightening may be performed manually by pushing together the clamp arms, with subsequent locking by tightening of a bolt. Tightening and locking is preferably facilitated by complementary ratchet tooth arrangements on the two clamp arms.

Other examples of bony structures that a clamp of the present invention 10, 10A may be used for clamping, besides spinous processes, include transverse processes (TP), pedicles, etc. Accordingly, it should be understood that even though bony structures in this patent application have been labeled SP1 or SP2 for convenience (since one most preferred embodiment of the clamp or method herein is engagement of spinous processes), that label is not intended to limit the term “bony structure(s)” in this patent application to spinous processes. In FIG. 19, a clamp 10 of the present invention is clamping two adjacent transverse processes TP1, TP2. FIGS. 19-20 show that first and second structures 20, 30 (in this case hooks 20, 30) may lie on a first plane FP (which is a “first vertical plane” when the clamp is deployed) and the pivot point PV of body spacer 40 may lie on a more distal plane DP in relation to a user standing behind the posterior of the patient/subject. This more distal plane shown in FIG. 20 may be offset in relation to the first plane FP that hooks 20, 30 may lie on. In this embodiment, the offset of the pivot point is in the anterior direction anatomically (as is the case of the clamp clamping the spinous processes in FIG. 18). First structure 20 may be configured to and may exert force against the first bony structure SP1 at a first point of contact and likewise second structure 30 may be configured to and may exert force against a second bony structure SP2 at a second point of contact. In FIGS. 19-20 the pivot point, although not designated as such, lies on the distal plane DP which can be seen to be offset in relation to a line (on first plane FP) between first and second structures 20, 30 and in particular relative to a line between inside surfaces of first and second structures 20, at first and second points of contacts PC1, PC2 of first and second structures 20, 30.

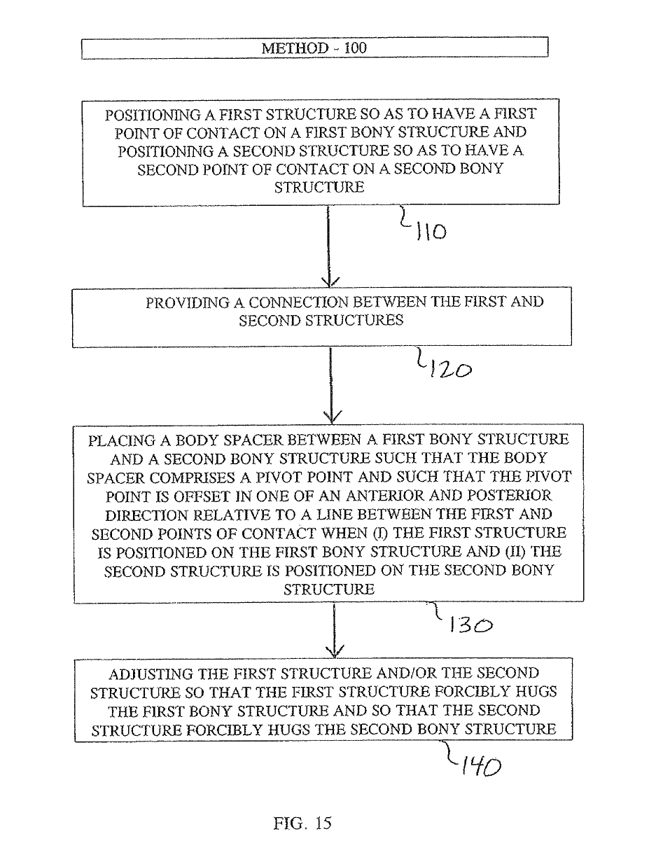

Accordingly, as shown by the flow chart of FIG. 15, the present invention may also be described as a method 100 of clamping bony structures. The following steps are not intended to be limited to the order presented and the steps may be implemented in a different order in other preferred embodiments. Method 100 may include a step 110 of positioning a first structure (such as a first hook) so as to have a first point of contact on a first bony structure, such as a spinous process (for example an upper part of the first spinous process), and positioning a second structure (such as a second hook) so as to have a second point of contact on a second bony structure, such as a spinous process (for example a lower part of the second spinous process). A further step 120 may involve providing a connection between the first and second structures, such as by providing a connecting rod between the hooks 20, 30 or by providing a connection between a rod projecting from one of the first and second structures 20, 30 to the other of the first and second structures 20, 30, or else by means of a clamping structure (which may be integrally connected to the clamp or may be an external device).

Method 100 may further include a step 130 of placing a body spacer 40 between a first bony structure and a second bony structure such that the body spacer comprises a pivot point PV and such that the pivot point PV is offset in an anterior direction (or in certain other preferred embodiments in a posterior direction) relative to a line between the first and second points of contact when the first and second structures 20, 30 (such as hooks) are respectively positioned to engage, i.e. have a point of contact on such to clamp, the first and second bony structures. An alternative to step 130 may involve placing a body spacer 40 between a first bony structure and a second bony structure such that the body spacer comprises a pivot point PV and such that the pivot point PV is offset in an anterior direction (or alternatively in a posterior direction) relative to lines of force from the first and second structures 20, 30.

As far as the degree of offset is concerned relative to the line L connecting the points of contact (or points on such a line L) or relative to the lines of force exerted by the hooks 20, 30, the pivot point PV of spacer pivot 40 may be offset relative to the line or line of force by at least a half a centimeter, or in other preferred embodiments, by between half a centimeter and two centimeters, or in still other preferred embodiments, by between half a centimeter and one and a half centimeters.

Another step 140 of method 100 may be adjusting the first structure 20 to forcibly hug the first bony structure and adjusting the second structure 30 to forcibly hug the second bony structure. Step 140 may more generally be expressed as adjusting the first structure and/or the second structure so that the result is that the first structure forcibly hugs the first bony structure and so that the second structure forcibly hugs the second bony structure. The first and second hooks 20, 30 may respectively hug the first and second bony structures at far ends of the first and second bony structures, proximal to the user.

Method 100 may have other steps. For example, there may be a step of positioning the first and second hooks by rotating the first and second hooks, in some preferred embodiments. The rotating may be 90 degrees from one vertical plane to another vertical plane, for example from a sagittal plane to a frontal plane. In other preferred embodiments, the rotating may be from a plane that is substantially vertical (within 10 rotational degrees) to another plane that is substantially vertical. Method 100 may have a step of adjusting a distance between the first and second hooks using a ratchet and tooth mechanism. In other preferred embodiments, method 100 may involve adjusting a distance between the first and second hooks by having a rod connecting the first and second hooks, wherein the first hook is connected to the rod by a first axis, the first hook rotatable around the first axis, and wherein the second hook is connected to the rod by a second axis, the second hook rotatable around the second axis and wherein the rod has a slot to which the first and second axis are connected, the first and second axis movable relative to one another within the slot.

Method 100 may also have a step of deploying onto a bony structure one of the first and second structures 20, 30 that has a rod projecting therefrom. Method 100 may also have a step of using the rod to set a position of the one of the first and second structure and to set a position of the body spacer.

In some preferred embodiments, method 100 may have one or more of the following steps: providing the other one of the first and second structures (the one that does not have a rod projecting therefrom) with a polyaxial head that may have a concavity (i.e. a tulip type head); providing the body spacer with a locking plate that may have a concavity (or may have an adjacent concavity); and using the rod projecting from the one of the first and second structures to set the position of the first and second structures and the body spacer by situating the rod into the polyaxial head of the other one of the first and second structures and into the concavity of the (or adjacent to) locking plate of the body spacer.

There may also be a step of locking the body spacer with a locking screw 43.

Method 100 may also have a step, in some embodiments, of inserting the first structure in a straightened state, the first structure comprising a plurality of segments hingedly interconnected, a first channel passing from a distal one of said segments along a majority of a length of the plurality. A further step using this technology may involve using a tightening element passing along the first channel to bias the first hook to a curved deployed state for hugging the first bony structure. Furthermore, there may be a step of inserting the second hook in a straightened state, the second hook comprising a plurality of segments hingedly interconnected, a second channel passing from a distal one of said segments along a majority of a length of the plurality. This technology may further have a step of using a tightening element passing along the second channel to bias the second hook to a curved deployed state for hugging the second bony structure.

According to one preferred but non-limiting embodiment, the body spacer 40 is first introduced between the bony structures. The proximal segment (if the body spacer is comprised of segments) or portion of the body spacer 40 may hold a locking mechanism. First and second structures 20, 30 may then placed on the first and second bony structures, for example first and second spinous processes, above and below the spacer 40. First and second structures 20, 30, which may be hooks, are then rotated 90 degrees to fit into the lock. The first and second structures 20, 30 may then be forcibly set (for example by an approximating tool placed on the 90 degrees bend in each hook) at an appropriate degree of compression to firmly hug the bony structures above and below the spacer 40. If a ratchet and tooth mechanism is utilized, the first and seconds structures 20, 30 may be gradually adjusted along the ratchet arrangement on the proximal ends of the first and second structures (i.e. along the rods 50a, 50b projecting from the first and second structures 20, 30). A locking screw of the lock may be locked to preserve the compression achieved.

The clamp of the present invention, and any of its components, including for example the first and second structures 20, 30 and the body spacer 40, may be made from any appropriate bio-compatible material, such as PEEK, titanium, steel, etc. In the case of the non-linear technology for the embodiment shown in FIGS. 13-14 based on the technology incorporated from the references patent publication 2010/0198263, the first and second structures may optionally be made from shape memory alloy or any of the other materials cited in this referenced publication.

While the invention has been described with respect to a limited number of embodiments, it will be appreciated that many variations, modifications and other applications of the invention may be made. Therefore, the claimed invention as recited in the claims that follow is not limited to the embodiments described herein.

1. A clamp comprising:
a first structure configured to exert force on a first bony structure at a first point of contact;
a second structure configured to exert force on a second bony structure at a second point of contact; and
a spacer pivot configured to be positionable between the first and second bony structures and comprising a pivot point offset in one of an anterior and posterior direction relative to a line between the first and second points of contact when (i) the first structure is deployed to engage the first bony structure and (ii) the second structure is deployed to engage the second bony structure,
the first and second structures connected one another directly or through an intermediate structure,
the spacer pivot operatively connected to the first and second structures when the first and second structures are deployed at the respective first and second points of contact and the spacer pivot is positioned between the first and second bony structures.

2. The clamp of claim 1, wherein the first structure is a first hook and the second structure is a second hook.

3. The clamp of claim 1, wherein a clamping structure connects the first and second structures.

4. The clamp of claim 3, wherein the spacer pivot is connected to the clamping structure.

5. The clamp of claim 1, further comprising a clamping structure configured to clamp the first structure to the first bony structure and the second structure to the second bony structure, the clamping structure configured to adjust a distance in at least one dimension between the first and second structures, the spacer pivot connected to the clamping structure and/or to at least one of the first and second structures.

6. The clamp of claim 1, wherein the first bony structure is a first spinous process and the second bony structure is a second spinous process and wherein the pivot point is offset in the anterior direction relative to the line between the first and second points of contact.

7. The clamp of claim 1, wherein the first bony structure is a first spinous process and the second bony structure is a second spinous process and wherein the pivot point is offset in the anterior direction relative to the line between the first and second points of contact.

8. The clamp of claim 1, wherein the clamp is adapted to clamp adjacent vertebrae at a lordotic angle.

9. The clamp of claim 1, wherein the clamp is adapted to clamp adjacent vertebrae at a kyphotic angle.

10. (canceled)

11. The clamp of claim 1, wherein the pivot point is offset relative to the line by between half a centimeter and two centimeters.

12. (canceled)

13. The clamp of claim 1, wherein a rod connects the first and second structures, and wherein each of the first and second structures is movable and lockable in place after positioning.

14. (canceled)

15. (canceled)

16. The clamp of claim 1, wherein the clamping structure includes a rod connecting the first and second structures and wherein the rod includes a ratchet and tooth mechanism enabling the first and second structures to move relative to one another and to lock in place.

17. The clamp of claim 1, wherein one of the first and second structures is integrally joined to a rod and another of the first and second structures has a head, the head having a concavity, the body spacer also having a locking plate, the locking plate having a concavity, the rod configured to fit into the concavity defined by the heads of the another of the first and second structures and into the concavity of the locking plate.

18. The clamp of claim 1, wherein each of the first and second structures comprises a curved gripping part, each said curved gripping part comprising
a plurality of segments hingedly interconnected so as to assume a straightened state for insertion and a curved deployed state for use in the clamp, a channel passing from a distal one of said segments along a majority of a length of the plurality of segments.

19. The clamp of claim 18, wherein each of the first and second structures also comprises an elongated tightening element anchored at the distal segment and passing along the channel such that tension applied to the tightening element tends to bias the plurality of segments from the straightened state to the curved deployed state.

20. A method of clamping bony structures, comprising:
positioning a first structure so as to have a first point of contact on a first bony structure and positioning a second structure so as to have a second point of contact on a second bony structure;
placing a body spacer between a first bony structure and a second bony structure such that the body spacer comprises a pivot point, such that the pivot point is offset in one of an anterior and posterior direction relative to a line between the first and second points of contact when (i) the first structure is positioned on the first bony structure and (ii) the second structure is positioned on the second bony structure and such that a connection is maintained between the first and second structures; and
adjusting the first structure and/or the second structure so that the first structure forcibly hugs the first bony structure and so that the second structure forcibly hugs the second bony structure.

21. The method of claim 20, further comprising utilizing a first structure that has a first hook and utilizing a second structure that has a second hook, and further comprising positioning the first and second hooks by rotating the first and second hooks.

22. (canceled)

23. The method of claim 20, further comprising utilizing a first structure that has a first hook and utilizing a second structure that has a second hook, and further comprising the first and second hooks respectively hugging the first and second bony structures at far ends of the first and second bony structures.

24. The method of claim 20, further comprising adjusting a distance between the first and second hooks using a ratchet and tooth mechanism

25. The method of claim 20, further comprising adjusting a distance between the first and second structures by either having a rod connecting the first and second structures or by using a rod projecting from one of the first and second structures to connect to the other of the first and second structures.

26. (canceled)

27. The method of claim 20, further comprising
deploying on a bony structure one of the first and second structures, the first structure being a first hook and the second structure being a second hook; and
using the rod to set a position of the first structure and the second structure and the body spacer.

28. The method of claim 20, wherein the first structure is a first hook and the second structure is a second hook and further comprising
providing a rod integrally joined to one of the first and second hooks;
providing the other one of the first and second hooks with a head having a concavity;
providing the body spacer with a locking plate having a concavity; and
using the rod to set the position of the first and second hooks and the body spacer by situating the rod into the concavity of the head of the other one of the first and second hooks and into the concavity of the locking plate.

29. The method of claim 28, further comprising locking the body spacer with a locking screw.

30. The method of claim 20, further comprising:
inserting the first structure in a straightened state, the first structure comprising a plurality of segments hingedly interconnected, a first channel passing from a distal one of said segments along a majority of a length of the plurality;
using a tightening element passing along the first channel to bias the first structure to a curved deployed state for hugging the first bony structure;
inserting the second structure in a straightened state, the second structure comprising a plurality of segments hingedly interconnected, a second channel passing from a distal one of said segments along a majority of a length of the plurality;
using a tightening element passing along the second channel to bias the second hook to a curved deployed state for hugging the second bony structure.

31. (canceled)

32. The method of claim 20, further comprising placing the body spacer such that the pivot point is offset relative to the line by between half a centimeter and two centimeters.

33. (canceled)

34. A clamp comprising:
a first structure configured to exert force on a first bony structure at a first point of contact;
a second structure configured to exert force on a second bony structure at a second point of contact; and
a spacer pivot configured to be positionable between the first and second bony structures and comprising a pivot point, the pivot point offset in one of an anterior and posterior direction relative to a line of force exerted by the first structure and relative to a line of force exerted by the second structure, when (i) the first structure is deployed to engage an upper part of the first bony structure and (ii) the second structure is deployed to engage a lower part of the second bony structure,
the first and second structures connected to one another directly or through an intermediate structure,
the spacer pivot operatively connected to the first and second structures when the first and second structures are deployed at the respective first and second points of contact and the spacer pivot is positioned between the first and second bony structures.

35. (canceled)
